(19) United States
(12) Patent Application Publication
(10) Pub. No.: US 2006/0259026 A1
(43) Pub. Date: Nov. 16, 2006

(54) ELECTROSURGICAL TREATMENT METHOD AND DEVICE

(75) Inventors: Neil Godara, Mississauga (CA); Taylor Hillier, Georgetown (CA); Amanda Martyn, Toronto (CA); Nir Lifshitz, North York (CA); Krishan Shah, Mississauga (CA)

Correspondence Address: DIMOCK STRATTON LLP, 20 QUEEN STREET WEST SUITE 3202, BOX 102, TORONTO, ON, M5H 3R3, CA

(73) Assignee: Baylis Medical Company Inc., Montreal (CA)

(21) Appl. No.: 11/381,783

(22) Filed: May 5, 2006

Related U.S. Application Data
(63) Continuation-in-part of application No. 11/280,604, filed on Nov. 15, 2005.
(63) Continuation-in-part of application No. 11/356,706, filed on Feb. 17, 2006.
(60) Provisional application No. 60/594,787, filed on May 5, 2005.
(60) Provisional application No. 60/595,426, filed on Jul. 4, 2005.
(60) Provisional application No. 60/595,559, filed on Jul. 14, 2005.
(60) Provisional application No. 60/595,560, filed on Jul. 14, 2005.

(51) Int. Cl. A61B 18/14

(52) U.S. Cl. 606/41; 607/99

(57) ABSTRACT

A method and device are disclosed for performing an electrosurgical procedure on a bodily tissue. The device has insulated and conductive regions for creating lesions in bodily tissue. A method of using the device is also described, including specific methods of diagnosing and treating sacroiliac-related pain using the device.

### REFERENCES TO PARENT AND CO-PENDING APPLICATIONS

This application claims priority from and is a continuation-in-part of co-pending U.S. patent application Ser. No. 11/280,604, filed Nov. 15^{th}, 2005, and Ser. No. 11/356,706 filed Feb. 17^{th}, 2006. In addition, this application claims the benefit of: U.S. provisional application No. 60/594,787, filed May 5^{th}, 2005; U.S. provisional application No. 60/595,426, filed Jul. 4^{th}, 2005; U.S. provisional application No. 60/595,559, filed Jul. 14^{th}, 2005; and U.S. provisional application No. 60/595,560, filed Jul. 14^{th}, 2005. The aforementioned applications are all incorporated herein by reference.

### TECHNICAL FIELD

This invention relates to a method and device for electrosurgery, and more specifically to a method and device for creating lesions in tissue.

### BACKGROUND OF THE ART

Ferrante et al. (Radiofrequency Sacroiliac Joint Denervation for Sacroiliac Syndrome; Regional Anaesthesia and Pain Medicine, Vol. 26, No. 2, pp. 137-142, March-April 2001), which is incorporated herein by reference, describe the creation of a strip lesion along the long axis of the posterior sacroiliac (SI) joint using Radiofrequency (RF) energy. Multiple probes are inserted along the joint margin and energy is delivered in a bipolar configuration. Such an approach requires multiple probe insertions and requires relatively precise probe placement in order to ensure adequate lesioning between the bipolar probes. Gevargez et al. (CT-Guided Percutaneous Radiofrequency Denervation of the Sacroiliac Joint; Eur Radiol (2002) 12:1360-1365), which is incorporated herein by reference, describe the creation of a strip lesion through the interosseous ligament surrounding the SI joint using RF energy. The approach detailed therein requires multiple energy delivery and repositioning steps and does not allow for the creation of a lesion within the intra-articular space of the SI joint itself. Yin et al. (Sensory Stimulation-Guided Sacroiliac Joint Radiofrequency Neurotonomy: Technique based on Neuroanatomy of the Dorsal Sacral Plexus; (2003) SPINE, Vol. 28, No. 20, pp. 2419-2425), which is incorporated herein by reference, advocate lesioning a single branch of a sacral nerve as it exits the sacral foramina. The procedure described by Yin et al. may require a relatively skilled user due to the approach involved. In addition, the procedure detailed therein is time consuming as it involves multiple steps of probe re-positioning and neural stimulation in order to locate a single symptomatic nerve branch. Furthermore, this procedure does not allow for the creation of a strip lesion nor does it allow for the creation of a lesion within the SI joint. Thus, it would be desirable to have a procedure to treat the SI region using energy delivery that overcomes some or all of the limitations of the prior art.

### BRIEF DESCRIPTION OF THE DRAWINGS

In order that the invention may be readily understood, embodiments of the invention are illustrated by way of examples in the accompanying drawings, in which:

FIG. 1 is an illustration of the location of a sacral neural crescent in a patient's body;

FIG. 2 shows a partial side elevation view of an embodiment of a probe having a continuously conductive current-delivering portion as compared to a probe having a discontinuously conductive current delivering portion, along with an example of a temperature distribution that may result from the delivery of energy from the probe in each case;

FIG. 3 shows a top plan view of an embodiment of an electrosurgical device of the present invention;

FIG. 4A shows a side elevation view of a distal portion of an embodiment of an electrosurgical device of the present invention;

FIG. 4B shows a top plan view of the embodiment of FIG. 4A;

FIG. 4C shows a bottom plan view of the embodiment of FIG. 4A;

FIG. 5 is a side sectional view of a distal portion of the device shown in FIG. 4A;

FIGS. 6A and 6B show a current-delivering window in accordance with an embodiment of the present invention;

FIG. 6C shows a side elevation view of a current-delivering window in accordance with a further embodiment of the present invention;

FIG. 6D shows a partial side elevation view of a further embodiment of an electrosurgical device of the present invention;

FIG. 7 shows a side elevation view of a distal portion of the embodiment of FIG. 4, showing an example of a lesion formed along the device;

FIG. 8 is a sectional end view through the device shown in FIG. 5;

FIG. 9A shows a side elevation view of a distal portion of an alternate embodiment of an electrosurgical device of the present invention;

FIG. 9B is a side sectional view of a distal portion of the device shown in FIG. 9A;

FIGS. 10A and 10B show side elevation views of distal portions of alternate embodiments of an electrosurgical device of the present invention;

FIG. 11 shows a side elevation view of a further embodiment of an electrosurgical device of the present invention;

FIGS. 12A and 12B show side elevation views of embodiments of a curved electrosurgical device of the present invention; and

FIG. 13 shows a rear elevation view of the sacro-iliac region of a human.

### DETAILED DESCRIPTION OF THE INVENTION

As used herein, the term ‘sacroiliac region’ refers to the region of the patient's body comprising the sacrum and ilium and their articulation (including the sacroiliac joints) or associated ligaments.

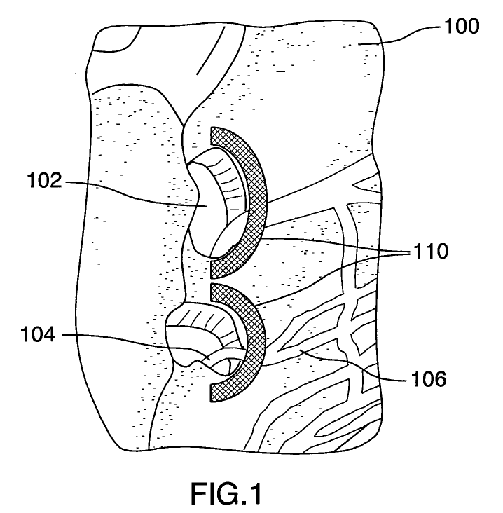

Furthermore, as used herein, the ‘sacral neural crescent’ refers to an area lateral to each of the sacral foramina, through which the sacral nerves are believed to pass after exiting the foramina. On the dorsal right side of the sacrum, this window is from about 12 o'clock to about 6 o'clock in a clockwise direction, while on the dorsal left side of the sacrum the window is from about 6 o'clock to about 12 o'clock in a clockwise direction. Similar (but in the counter-clockwise direction) areas exist on the ventral side of the sacrum. The clock positions are referenced as if the foramen is viewed as a clock face, and the view is taken looking towards the sacrum. For reference, the 12 o'clock position of the clock face would be the most cephalad (towards the head) point of the foramen. FIG. 1 illustrates the position of two sacral neural crescents 110 on the dorsal right side of the sacrum 100. As can be seen, sacral nerves 104 and lateral branches 106 exit each of the sacral foramina 102 and pass through sacral neural crescents 110.

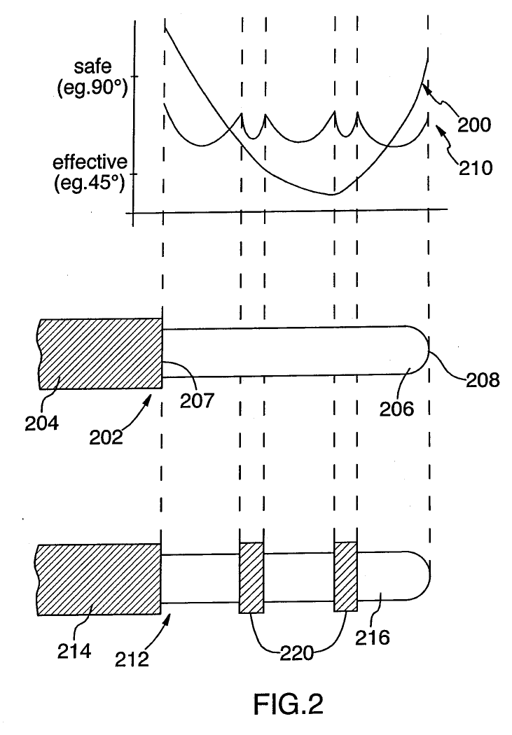

As used in the present description, a substantially homogeneous lesion may refer to a continuous lesion that is created along the length of the conductive portion of a probe such that substantially all portions of tissue within the lesion area are at a temperature within a given range, where the range lies between an efficacious temperature (at the low end) and a safe temperature (at the high end). For example, if tissue must be heated to about 45 degrees Celsius in order to create a lesion effective to treat a tissue, while anything above about 90 degrees Celsius may be dangerous and damaging to the tissue, a substantially homogeneous lesion may be a continuous lesion within which substantially all portions of tissue have a temperature between about 45 to about 90 degrees Celsius. This concept is illustrated in FIG. 2, which shows probes 202 and 212 and plots 200 and 210 of potential temperature distributions for tissue within a lesion created along the lengths of the respective probes. Thus, as shown in FIG. 2, temperature distribution 200 may be indicative of a lesion which is not substantially homogeneous while temperature distribution 210 may be indicative of a lesion which is substantially homogeneous. In conjunction with the understanding of a substantially homogeneous lesion, the phrase ‘Substantially homogeneous energy delivery’ may be understood to describe the delivery of energy such that a substantially homogeneous lesion is created.

In the context of the present description, the terms ‘ablation’ and ‘lesion,’ amongst others, are used to indicate that a treatment effect has been produced. Thus, usage of these and similar terms are not intended to limit the application of the present invention to a specific mode of action or treatment effect but are rather intended to illustrate certain particular effects that may be achieved through practicing the present invention.

Furthermore, any reference to an ‘insulator’, ‘insulating material’ or ‘insulated region’, in the context of the present description, refers to a material or region that is at least partially electrically insulating (unless otherwise stated), although it may, depending on the application, be thermally insulating as well.

In addition, in the context of the present description, the term ‘probe’ is used to describe any elongate device that may be percutaneously inserted into a patient's body. These devices include but are not limited to catheters, cannulae and electrosurgical probes. For the sake of clarity, the term ‘probe’ is used throughout the description to describe any such device. The device can be flexible, stiff or semi-rigid or any combination thereof, and the invention is not limited in this regard. Similarly, any references to an introducer, sheath, cannula or other medical device is not intended to be limited to a specific device. Rather, any device that may accomplish the same function as these devices is intended to be within the scope of the present invention.

In the description of the present invention, the term ‘proximal’ is used to refer to a portion or region of a device or tissue that is located closest to the user. ‘Distal’ refers to a portion or region of a device or tissue that is located closest to a treatment site and furthest away from the user.

An embodiment of the invention thus provides a method of delivering energy to a sacroiliac region of a patient's body, through a device having at least one electrically insulated region along a portion of a circumference of the device and at least one current delivering region along a remaining portion of the circumference of the device comprising, in any order, the steps of: positioning the electrically insulated region and the current delivering region within the sacroiliac region of a patient's body such that the current delivering region is electrically exposed to a target site and at least a portion of the electrically insulated region shields another site; and delivering energy to the device, whereby energy is delivered substantially towards the target site and substantially prevented from being delivered to the shielded site.

With specific reference now to the drawings in detail, it is stressed that the particulars shown are by way of example and for purposes of illustrative discussion of certain embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Before explaining the structure in detail, it would be beneficial to illustrate a proposed theoretical concept underlying the function of the present invention. It is important to note that, although the theory being presented is currently believed to be true and accurate, the invention should not be limited by this or any other theory of operation. Rather, the breadth of the invention as will be presently described is intended to be limited only by the scope of the appended claims.

The distribution of electrical current, or current density, arising from a radiating source such as an electrical conductor, is governed by several mathematical formulae, including Laplace's equation and Maxwell's equations. It is postulated that when an electrical conductor has regions of abrupt disruptions of conductivity (i.e. electrical discontinuity), these equations dictate that the current density in the vicinity of those regions will be higher than the current density found around other regions of the conductor. The term ‘electrical discontinuity’ may refer to any feature of a probe or other electrical device that may affect energy delivery from the surface of the probe to a surrounding environment. For example, an insulating material coating one or more regions of the active portion of a probe would result in an electrical discontinuity since energy delivery from the portion of the probe located around the insulating material is affected by the insulating material. Furthermore, notches or other surface irregularities in the active portion may also be considered to be electrical discontinuities, since they also may affect the delivery of energy from the surface of the probe to the surrounding environment. As another example, a distal end of a probe is considered to be an ‘electrical discontinuity’ if there is a disruption of energy delivery from the surface of the probe to the surrounding environment about the distal end of the probe.

Phenomena resulting from disruptions in conductivity may be referred to as ‘edge effects’ and regions of high current density may be referred to as ‘hot spots’. So, for example, the current density in the vicinity of an interface between a conductive region and an electrically insulated region of an electrosurgical probe, may be higher than in the vicinity of the continuously conductive region of the probe, resulting in a localized ‘hot spot’ near the insulator/conductor interface. The temperature of tissue in the vicinity of a certain region of the probe may be proportional to the current density in that region.

FIG. 2 shows the typical temperature distribution 200 that may be achieved using an electrical probe 202 with an insulated region 204 and a continuous conductive region 206. Two ‘hot spots’ of increased temperature, typically located at the two edges 207, 208 of conductive region 206 of the device, are shown. The temperature decreases gradually between the edges 207, 208, generally achieving a minimum value near the middle of conductive region 206. FIG. 2 also shows by way of comparison an electrical probe 212 with an insulated region 214 and a current-delivering region comprising a conductive body 216 that is surrounded by a plurality of insulating bands 220 which interrupt the radiation of current. This configuration would generally lead to the temperature distribution 210, wherein a ‘hot spot’, or local temperature maximum, is generated in the vicinity of every interruption in current radiation. Where the insulated parts of the probe 212 are properly spaced, this configuration spreads out the temperature distribution more evenly along the length of conductive body 216, resulting in a continuous and more substantially homogeneous lesion if sufficient power is supplied to probe 212.

The longer the distance of the conductive region between subsequent insulating bands, the more variation there will be in current density along that conductive region and the less radially uniform the lesion will be. Similarly, if the axial lengths of insulating bands 220 are increased, a discontinuity in the lesion formed may result due to the fact that there is virtually no current radiation surrounding the insulating bands and in this area lesion formation is entirely dependent upon thermal conduction effects. If the bands are relatively short axially, the current radiating from the electrically conductive regions immediately adjacent to each insulated band will generate sufficient heat to cause a lesion to form across the insulated bands due to thermal conduction, but the more the lengths of the insulating bands are increased, the less uniform the lesion will be because thermal conduction will be less effective.

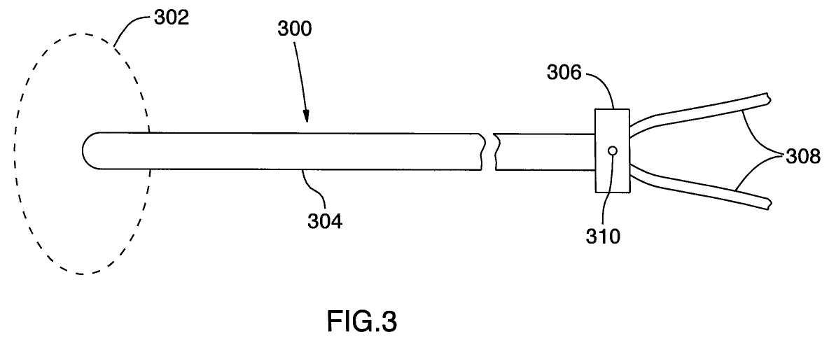

In one embodiment, as shown in FIG. 3, a device embodying the present invention may comprise a probe 300 for treating a tissue 302 in a body, wherein probe 300 comprises a shaft 304, a handle 306 and one or more connecting means 308.

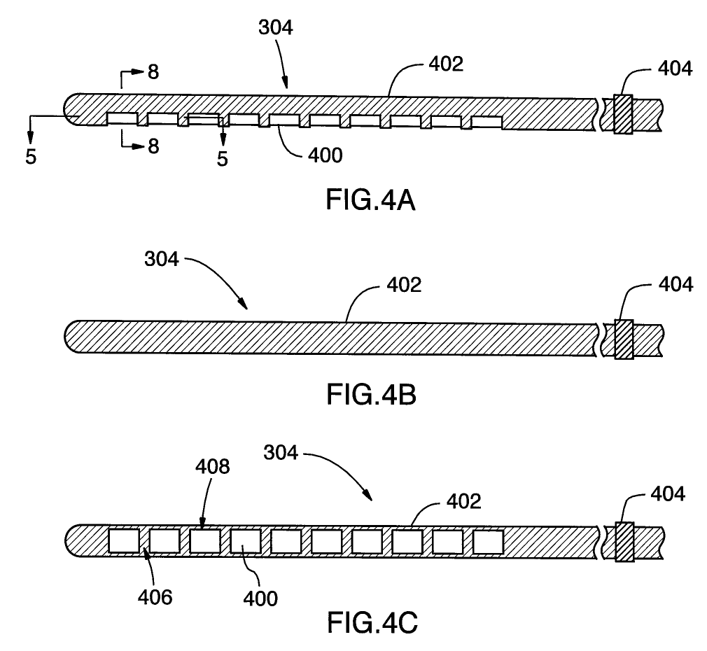

In this embodiment, shaft 304, shown in FIGS. 4A to 4C, comprises at least one conductive body 400 having a distal region and a proximal region, an insulating layer such as a dielectric or semi-conductive coating 402, and optionally one or more markings 404 to aid in positioning the device.

Conductive body 400 is electrically coupled, via connecting means 308, to an energy source (not shown). The energy source supplies high frequency electromagnetic energy (e.g. radiofrequency energy) to conductive body 400. Conductive body 400 may be coupled to the energy source in a monopolar configuration, whereby a return electrode, for example a grounding pad (not shown) having a relatively large surface area, is placed at a distance from probe 300, for example on a remote surface of the patient's body. Alternatively, conductive body 400 may be employed in a bipolar configuration, where an electrode having a relatively similar size is placed in or on the body near probe 300. Conductive body 400 may be rigid or flexible and may be straight, bent or angled at one or more points along its length. As used herein, the term ‘bent’ refers to any region of non-linearity or any deviation from a longitudinal axis. In addition, conductive body 400 may be made of various materials, including but not limited to stainless steels and/or shape memory alloys such as Nitinol (nickel-titanium).

In alternate embodiments, ultrasonic energy may be used. In such embodiments, the energy source may be operable to provide ultrasonic energy to at least one ultrasonic transducer located on the device. Some such embodiments may comprise one or more means for preventing delivery of ultrasonic energy to a non-target tissue site so that the ultrasonic energy can be directed towards a target tissue site, away from the non-target site. For example, one or more vibration dampeners may be used.

As shown in FIG. 4B, in this embodiment one circumferential region of shaft 304 may be substantially entirely insulated along its length. A remaining circumferential region of shaft 304 has bands 406 of insulating coating 402 with windows 408 along its length, exposing portions of the conductive body 400 along a current-delivering region, as shown in FIG. 4C. This may be achieved by providing an integral insulating coating 402 on shaft 304 such that current-delivering windows 408 remain electrically exposed, however it will be appreciated that these portions of the probe may be insulated separately, using the same or different materials, and by means of a dielectric or semiconductive coating, or by any other suitable means. In alternate embodiments, the remaining circumferential region of shaft 304 may comprise a substantially continuous electrically-exposed portion, for example without bands of insulating material. Thus, in either of these embodiments, the device has at least one electrically insulated region along a portion of a circumference of the device and at least one current delivering region along a remaining portion of the circumference of the device.

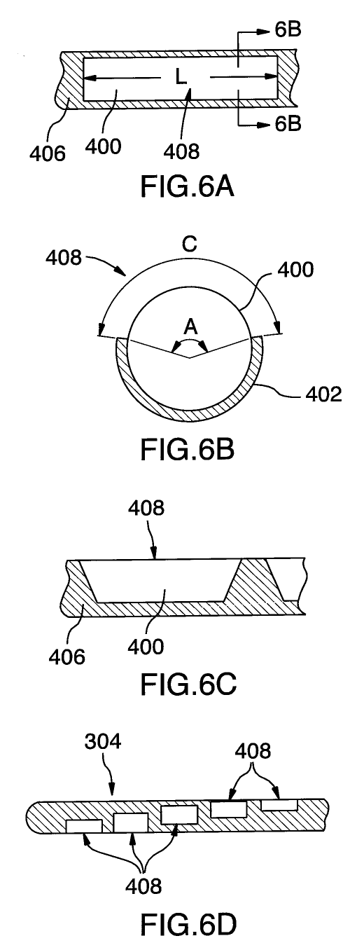

The shaft 304 in the device of the invention does not have to be cylindrical in cross-section, but can be oval, polygonal, or any other desired shape. As used in the present description, the term “circumferential region” refers to a region along a length of the shaft 304 that extends about a portion of the exterior of the shaft 304, but does not extend entirely about the shaft 304. For example, FIG. 6B shows a current-delivering circumferential region having an angle A° and an insulated circumferential region having an angle (360°-A°). It will be appreciated that this term applies equally to non-cylindrical shafts.

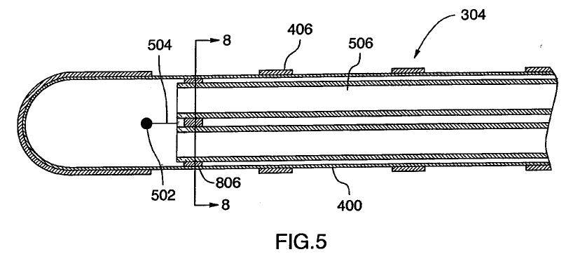

FIG. 5 shows a longitudinal section through a distal region of shaft 304. In this view it can be seen that shaft 304 may further optionally comprise one or more sensors 502, wiring 504 electrically insulated from the conductive body 400 to carry signals from the sensor 502 to a controller and/or a measuring device (not shown), and/or one or more internal lumens 506 which can serve a number of purposes, some of which are described below, including as a cooling means to circulate a coolant that reduces the temperature of the tissue in the vicinity of the shaft 304.

As shown in FIGS. 6A and 6B, current-delivering windows 408 through which portions of the conductive body 400 are in conductive communication with tissue are each defined by a gap in the insulating coating 402 having a longitudinal length L, and a circumferential width C defined by a sector of the conductive body 400 through an angle A. In this embodiment, current-delivering windows 408 may be between about 2 mm and about 5 mm in length (L) and may have circumferential widths (C) corresponding to angles (A) of between about 100 degrees to about 180 degrees. The insulating bands 406 between current-delivering windows 408 may have an axial length of between about 0.5 and about 1.5 mm. Any or all of these dimensions may vary.

Thus, current-delivering windows 408 and insulating bands 406 may have various dimensions, and any dimension of a current-delivering window 408 or insulating band 406 may vary along one of its other dimensions. For example, FIG. 6C shows a current-delivering window 408 having a length L that varies along its circumferential width C. The current-delivering windows 408 may also be irregularly shaped, and need not have the same shape as other current-delivering windows 408. Additionally, the current-delivering windows 408 do not need to be located along the same circumferential position of the shaft 304. For example, as shown in FIG. 6D, current-delivering windows 408 may have circumferential positions that vary along the length of shaft 304.

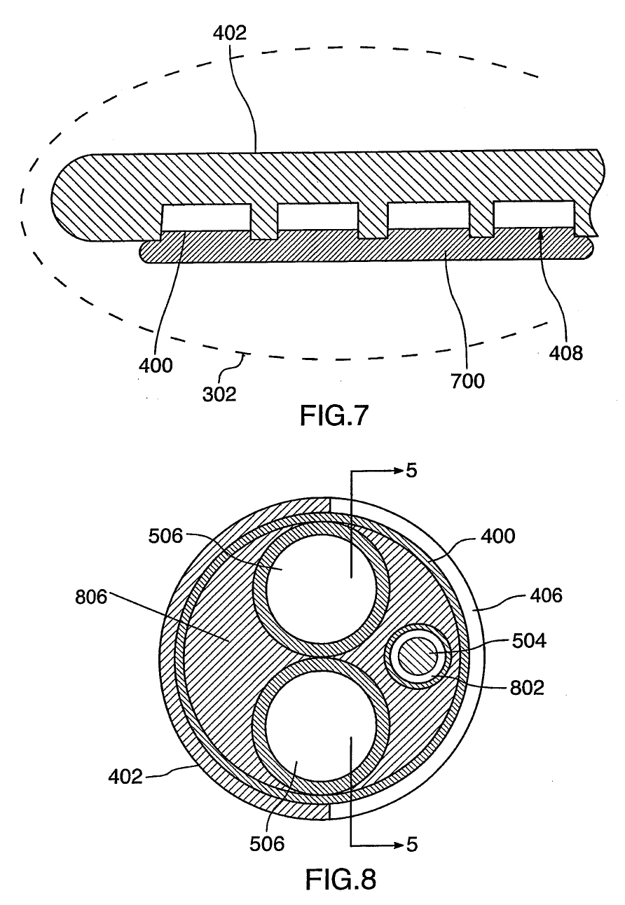

FIG. 7 illustrates a possible treatment effect that may result from the application of energy from the current-delivering portion of the probe 300 to tissue 302. Without being restricted or limited by a proposed theory of operation, it is suggested that the embodiment of the device described herein facilitates the creation of an elongated or strip lesion 700, for example a substantially homogeneous lesion, in tissue 302 in the vicinity of the current-delivering portion of the shaft 304, while inhibiting the creation of a lesion in the vicinity of other circumferential regions of the shaft 304. In other words, using a device described in this embodiment of the present invention, a lesion 700 may be formed along the portion of the probe 300 comprising current-delivering windows 408, while the creation of a lesion is inhibited along other portions of probe 300 that are substantially entirely insulated.

In addition, the lesion 700 that is created may take the form of an elongated or strip lesion due to the pattern of intermittent insulated and current-delivering regions, as has been described above. A more detailed description of probes comprising intermittent insulated and current-delivering regions is provided in U.S. patent application Ser. No. 11/356,706, filed on Feb. 17, 2006 and incorporated herein by reference. Furthermore, in embodiments of the present invention that comprise a cooled probe, such as that illustrated in FIGS. 4A to 4C and 5, the cooling may be effective to prevent the tissue immediately surrounding the probe 300 from reaching a lesioning temperature and thus cause the lesion to form at a distance from the probe, restricting the formation of a lesion even more to one side of the probe than to the other. Cooling a device to cause a lesion to form away from the probe is described in further detail in U.S. Provisional Patent Application 60/743,511, filed on Mar. 16^{th}, 2006, and U.S. Provisional Patent Application 60/595,560, filed on Jul. 14^{th}, 2005, both of which are incorporated herein by reference. Thus, a device of the present invention may utilize insulating material to both inhibit the creation of a lesion along one region of the device and to facilitate the creation of a lesion, for example a substantially uniform or homogeneous lesion, along another region of the device.

Markings 404 may include, but are not limited to, visual markings, tactile markings and radiopaque markings. Markings 404 may include more than one marking and markings 404 may be located at various locations of shaft 304. In addition, further markings may be located at various locations of probe 300, including, for example, a marking 310 along handle 306, as shown in FIG. 3. Marking 310 may be particularly useful to indicate one or more of the direction in which the current-delivering windows 408 are facing and/or the direction in which the probe is bent. Markings 404 may beneficially aid in positioning probe 300 within a patient's body and/or in determining the position of the current-delivering region within the patient's body. For example, markings 404 may comprise a radiopaque band and the radiopaque band may be located proximate the region of the current-delivering region of shaft 304, thus allowing a user to visualize, under fluoroscopic guidance, the area of tissue that may be treated by device 300. In a further embodiment, markings 404 may comprise a radiopaque marker at each current-delivering window 408 of shaft 304. Furthermore, a radiopaque stylet may be used to visualize a predicted size and location of a lesion, or to aid in positioning the probe, as is disclosed in U.S. Provisional Patent Application No. 60/744,518, filed on Apr. 10^{th}, 2006, which is incorporated herein by reference.

Sensors 502 may be used to monitor one or more of temperature, impedance, pressure, or any other property of tissue 302, probe 300, or a cooling system, for example. Temperature sensors may include thermocouples, thermistors, optical thermometers or other temperature sensing means. Shaft 304 may be furnished with at least one sensor 502 in the form of a thermocouple that may be formed, for example, by welding a wire 504 to conductive body 400, wherein wire 504 and conductive body 400 are made from different materials (e.g. constantan and stainless steel). There may be more than one sensor 502 present on shaft 304, for example, it may be beneficial to have a temperature sensor associated with each current-delivering window 408, or to have both a temperature sensor and an impedance sensor located at the distal end of shaft 304. In addition, it may be beneficial to have a sensor 502 projecting from shaft 304, in order to sense properties of tissue 302 at or near shaft 304, or it may be beneficial to have a sensor 502 recessed within shaft 304.

As shown in FIG. 5, shaft 304 may contain one or more lumens 506. Lumens 506 may be useful for a number of purposes, including but not limited to carrying fluid, isolating certain components of probe 300 such as electrical wires or guide wires, or for structural support. FIG. 8 shows an embodiment in which shaft 304 contains three lumens: one lumen 802 containing a wire, for example wire 504, and two lumens 506 for circulating a fluid, such as a cooling fluid which may be useful to improve the efficacy of a treatment procedure. In such an embodiment, shaft 304 may further contain a plug 806 for limiting the circulation of a fluid to a desired portion of shaft 304. Plug 806 may also act as a radiopaque marker if it is made of a suitable radiopaque material, such as silver solder.

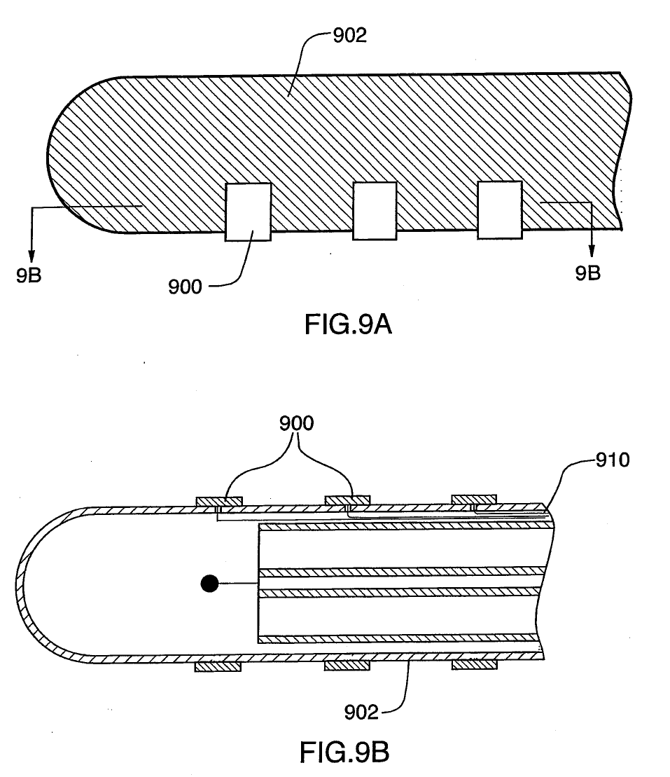

Wiring 504 may comprise, for example, wires associated with sensor 502 as well as any wires that may be associated with current-delivering windows. In some embodiments, for example as shown in FIGS. 9A and 9B, current-delivering windows 900 may be coupled to the energy source via one or more wires 910, rather than via shaft 902, which may be manufactured of material that is at least partially electrically insulating. In the illustrated embodiment, each current-delivering window 900 is effectively an electrically independent conductive body and may have a separate wire coupling it to the energy source. In this embodiment, the system may be operated in a multi-polar or bipolar mode, in which case windows 900 are electrically isolated from each other, or in a monopolar mode. In an alternate embodiment, current-delivering windows 900 may share wires such that each of the windows 900 is at the same electrical potential. Wiring 504 and 910 may be electrically insulated and may be made of various materials, depending on the application. For example, a constantan wire may be used as a component of a thermocouple, as described earlier.

Connecting means 308 may include any means of electrically coupling an energy source to probe 300 and more specifically to current-delivering windows 408. Thus, connecting means 308 may comprise one or more electrical cables or wires, as well as any necessary electrical connectors for coupling to an energy source. In embodiments wherein probe 300 is cooled, connecting means 308 may further comprise a means of connecting a coolant supply (not shown), for example a fluid delivery mechanism, to probe 300. For example, flexible tubing may be used, in conjunction with a connector such as a Luer-type connector, to connect a syringe or other fluid source to probe 300. Connecting means 308 may beneficially be flexible in order to allow for greater maneuverability of probe 300. Connecting means may be coupled to shaft 304 within handle 306.

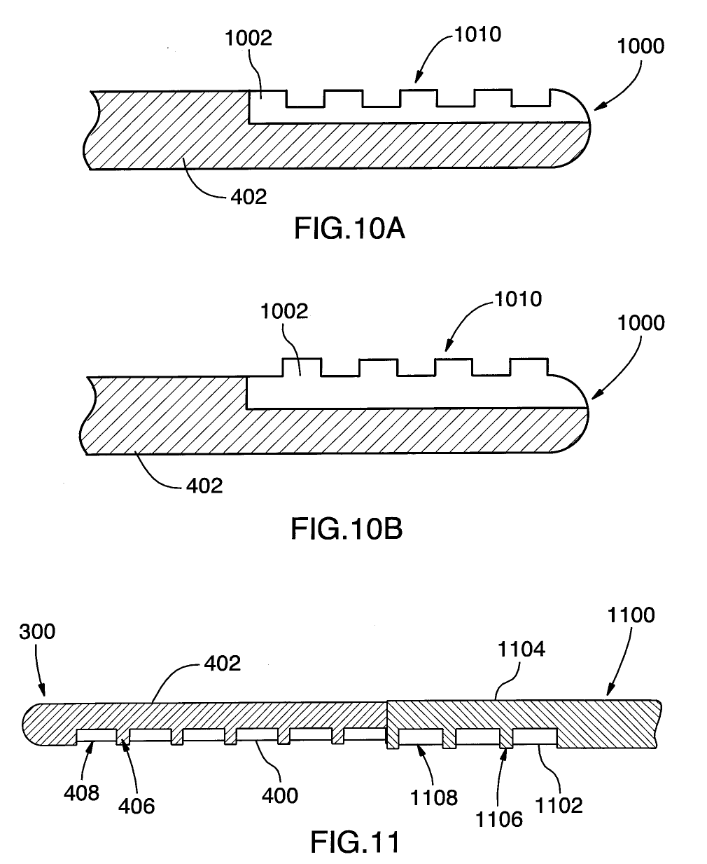

In an alternate embodiment, rather than having separate current-delivering windows along shaft 304, the surface of the electrically exposed or current-delivering region of shaft 304 may be modified in such a manner as to effectively create ‘edge effects’ as described above. For example, as shown in FIGS. 10A and 10B, shaft 1000 may have one or more portions 1010 along its length wherein material is removed in order to create abrupt electrical discontinuities. In some such embodiments, the electrically exposed region may be substantially corrugated, wherein bands of material of conductive body 1002 are removed or added to form a ribbed configuration. In alternate embodiments, portions of the surface of the electrically exposed region may be grit-blasted, resulting in bands of modified surface texture.

According to another embodiment of the present invention, the electrosurgical device may additionally comprise an introducer (in addition to probe 300), used to aid in the insertion of probe 300 into a body of a patient. FIG. 11 shows an embodiment of the invention using an introducer 1100, which could be, for example, a rigid stainless steel cannula. Alternatively, introducer 1100 may be bent and may be made of any of various materials or a combination thereof, including, for example, a shape memory alloy such as Nitinol (nickel-titanium). In the illustrated embodiment, introducer 1100 is made from a conductive material 1102 and has an insulating coating 1104, which may be made from the same material as insulating coating 402 on probe 300. The insulating coating 1104 may be distributed such that introducer 1100 has bands 1106 and current-delivering windows 1108, similar to probe 300. Furthermore, in some embodiments, introducer 1100 may be electrically coupled to an energy source, either directly or via probe 300. In such embodiments, a lesion may be formed along at least part of both introducer 1100 and probe 300 when energy is delivered from the energy source. Introducer 1100 may further comprise one or more sensors and/or one or more markings, as have been described with respect to probe 300 above. In addition, a distal end of introducer 1100 may be shaped so as to facilitate entry of introducer 1100 into a patient's body.

Various alternate embodiments of a device of the present invention are described below and, although they are described as separate embodiments, it is understood that they may be able to be combined (amongst themselves as well as with the aforementioned embodiments), and that such sub-combination embodiments are also considered to be included within the scope of the present invention.

In embodiments wherein conductive body 400 does not extend the entire length of shaft 304, conductive body 400 may be positioned at the distal end of shaft 304, at the proximal end of shaft 304, or at some other location on shaft 304, or more than one conductive body 400 may be used along shaft 304. In embodiments wherein conductive body 400 is positioned at least at the distal end of shaft 304, or extends the entire length of shaft 304, the distal tip of conductive body 400 may have any of a variety of shapes that are known in the art including, but not limited to: flat, rounded, pointed, beveled, concave, convex, or chisel-tipped. The distal end of conductive body 400 may be closed, or may be at least partially open, allowing communication between the interior of shaft 304 and tissue 302. Conductive body 400 may deliver alternate forms of energy including, but not limited to: microwave energy, ultraviolet energy, other frequencies of electromagnetic energy including optical energy, and thermal energy.

Insulating coating 402 may be made of any material with a lower conductivity than that of conductive body 400, including dielectric and semi-conductive materials, and may be applied to conductive body 400 using a variety of methods known in the art, including, but not limited to: placing a sleeve of insulating material overtop of conductive body 400 and applying energy to shrink the sleeve onto conductive body 400, applying a liquid coating to conductive body 400, and applying energy or performing some other treatment to a part of conductive body 400 in order to reduce its conductive properties. Current-delivering windows 408 in insulating coating 402 may be formed in a number of ways, for example, by being pre-formed or masked before application of insulating coating 402, or by selective removal of insulating material following application of the insulating material to conductive body 400. As mentioned above, current-delivering windows 408 need not be oriented along the longitudinal axis of shaft 304 and neither do they need to be positioned in a straight line or regularly along shaft 304. In one embodiment, current-delivering windows 408 are positioned in one orientation (for example, with their lengths extending circumferentially) at a distal region of shaft 304 and in another orientation (for example, with their lengths extending longitudinally) at a proximal region of shaft 304. In some embodiments, a distal tip of shaft 304 (for example, the distal-most 1 to 5 mm of shaft 304) is entirely uninsulated. Additionally, in some embodiments, one or more of current-delivering windows 408 and insulating coating 402 may not be affixed in a permanent position but their position may rather be adjustable, for example during the course of a treatment procedure.

Although the use of lumens 506 has been discussed with respect to previously-described embodiments of the invention, other embodiments exist wherein shaft 304 is solid, rather than hollow, thus having no internal lumens 506.

The embodiments above make reference to certain features of the device of the present invention. However, numerous additional features may be added to or used in conjunction with any or all embodiments of the device, as will be presently described.

In some embodiments, the device of the present invention may have a shaft 304 that is hollow. In these embodiments, shaft 304 may be at least partially occluded, either permanently or at some point in a treatment procedure, by a stylet, trocar or other occluding means. In one embodiment, shaft 304 is hollow and is open at the distal tip. In this embodiment, a stylet may extend through shaft 304 for at least partially occluding the open distal tip, which can aid in the insertion of the device into tissue 302.

Shaft 304 may be rigid or may have some degree of flexibility. Furthermore, shaft 304 may be steerable, manually deformable, or may be otherwise able to have its shape actively manipulated while within a body of a patient. In addition, shaft 304 may be made from a material with shape memory (e.g. Nitinol), such that it will adopt a predetermined shape when introduced into the body, without the need for active steering. Embodiments with steerable shafts 304 may employ, for example, guide wires, hinges, hydraulic means or electronic devices in order to push, pull, bend, curve, or otherwise change the shape or orientation of the device. Other embodiments may use materials that change shape when thermal or electrical energy is applied. In one embodiment, a guide wire is attached both to the distal end of shaft 304 and to an introducer 1100. As shaft 304 is advanced out of introducer 1100, tension is created in the guide wire, causing shaft 304 to change shape. In another embodiment, a guide wire attached to the distal end of shaft 304 is capable of being directly pulled or otherwise manipulated in order to exert pressure to cause a change in the shape or orientation of shaft 304. In another embodiment, multiple guide wires are attached to shaft 304, and are able to be manipulated in order to offer fine control over a change in shape or orientation of shaft 304. In this embodiment, shaft 304 may have multiple lumens 506, so that each guide wire passes through a different lumen 506. In one embodiment, the external surface of shaft 304 (which may comprise a conductive body 400 with an insulating coating) is at least partially threaded, as in a screw or drill-like device. In such an embodiment, the internal surface of introducer 1100 may also be threaded. The threading on either or both of introducer 1100 and shaft 304 may be irregular, such that rotation of shaft 304 within introducer 1100 causes shaft 304 to adopt a bend or curve as it exits introducer 1100. In embodiments wherein shaft 304 is steerable, shaft 304 may contain one or more rods or other elements that prevent movement, in order to hinder the movement or bending of shape 304 in a certain direction. In other embodiments, shaft 304 or conductive body 400 may be made from or overlaid with a spring mechanism in order to guide the direction or orientation of steering. In another embodiment shaft 304 may be made from a series of articulated segments that fit together so as to allow bending in at least one direction, while preventing bending in at least one other direction. In a further embodiment, shaft 304 is made from a flexible material, but is overlaid with a sheath made from articulated segments as described above.

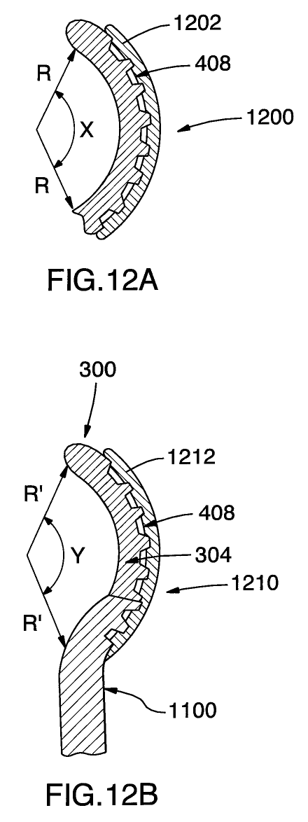

In embodiments wherein shaft 304 is pre-formed into a specific shape and is not otherwise actively steerable, shaft 304 may have various possible shapes, as has already been mentioned. For example, in one particular embodiment of the present invention, shown in FIG. 12A, a device 1200 as described with respect to the first embodiment may be pre-formed with a curve having a central angle X of between about 90 degrees and about 180 degrees, and, in some embodiments, between about 110 degrees and about 130 degrees, along with a radius of curvature R of between about 5 mm and about 15 mm. Such an embodiment may be particularly useful for treatment procedures involving a patient's sacrum, as will be discussed in greater detail with respect to a method aspect of the present invention. Briefly, it may be beneficial to create a lesion 1202 in the sacral neural crescent. In order to create such a lesion, a device 1200 of the present invention may be provided with a pre-formed curve, as has been described, such that current-delivering windows 408 may cover the desired treatment area.

In a second particular embodiment, shown in FIG. 12B, a device 1210 as described with respect to an alternate embodiment of the present invention may comprise a curved introducer 1100 as well as a probe 300 with a curved shaft 304. For example, the curve of introducer 1100 may cooperate with the curve of shaft 304 in order to cover an area of between about 110 degrees and about 130 degrees, with a radius of curvature R′ of between about 5 mm and about 15 mm, so as to be operable to create a desired lesion 1212 at the sacral neural crescent. For example, introducer 1100 and shaft 304 may each be curved by between about 15 and about 90 degrees such that, when curved shaft 304 is extended beyond a curved distal tip of introducer 1100, the total angle Y covered by the two curves is approximately 120 degrees. An exemplary device of this embodiment of the invention may comprise an introducer 1100 with a curve of approximately 30 degrees as well as a probe 300 comprising a shaft 304 having a curvature of approximately 90 degrees, with a radius of curvature for both the introducer and probe of approximately 10 mm. Alternatively, both introducer 1100 and probe 300 may be curved but the curves may not cooperate to cover a desired treatment area. Rather, introducer 1100 may be curved in a first direction to facilitate insertion and positioning within a patient's body while device 300 may be curved in a second, for example an orthogonal, direction in order to create a lesion over a desired area. In addition, probe 300 may be curved in more than one plane such that, upon extending probe 300 beyond a distal end of introducer 1100, a distal end of probe 300 may follow a corkscrew-like path (for example, down and to the side or up and to the side) in order to achieve a desired position within the body.

Lumens 506, when present, may be used for the circulation or delivery of any of a variety of fluids. In some embodiments, one or more lumens 506 are used to circulate a cooling fluid within probe 300; this fluid may be, for example, water, saline, ringer's solution, or any other fluid with sufficient viscosity and heat capacity to reduce the temperature of probe 300 or of tissue 302 upon circulation. In another embodiment, the distal tip of shaft 304 may be open or contain at least one aperture, and a cooling fluid may be released into a patient's body from shaft 304. In some embodiments, shaft 304 may contain one or more apertures along its length sufficiently sized to allow the release of some fluid from one or more lumens 506, while maintaining a fluid pressure high enough that some fluid is circulated back to a fluid delivery mechanism through one or more connecting means 308. Other fluids that may be circulated or delivered through lumens 506 include, but are not limited to, anesthetic fluid, electrically conducting fluid, pharmacological agent, contrast solution or other treatment fluid. A fluid delivered may have multiple desirable properties, for example, by being both cooled and having anesthetic properties.

As mentioned above, shaft 304 may have one or more apertures. The aperture(s) may lie on any part of the surface of the shaft, and may have any of a variety of shapes including, but not limited to: rounded, elongated, or rectangular. In addition, the edges of any or all apertures may be smooth in order to reduce the trauma caused to a patient's body during insertion of the device.

Various functional elements may provide additional functionality and are intended to be within the scope of the present invention. Such functional elements include, but are not limited to, elements for adding or removing material, elements for modifying tissue, and elements for visualizing tissue.

In any or all of the embodiments of the invention, various dimensions of the device, including but not limited to the diameter/gauge, the length and the radius and angle of curvature of any components of the device may vary, depending on the specific application and treatment procedure.

In a broad aspect, methods of the present invention allow for the delivery of energy to a target site, while preventing the delivery of energy to, or preventing the delivery of energy in whole or in part to another, non-target, site. As a feature of this aspect, the delivery of energy to the target site may involve the formation of a strip-lesion or a substantially homogeneous lesion at the target site. In general, method embodiments of the present invention may involve delivering energy to a region of a patient's body through a device having at least one electrically insulated region along a portion of a circumference of the device and at least one current delivering region along a remaining portion of the circumference of the device. The method embodiments may further comprise a step of positioning the electrically insulated region and the current delivering region within the region of the patient's body such that the current delivering region is electrically exposed to the target site and at least a portion of the electrically insulated region shields the other site. These method embodiments may allow energy to be delivered substantially towards the target site while preventing energy from being delivered to the other, shielded, site. As described herein, energy delivery may be effective to alter the structure or function, or both, of neural tissue. In some embodiments, sufficient energy is delivered in order to ablate one or more neural structures.

Broadly speaking, a method aspect of the invention describes a process for treating tissue that includes: inserting a device into a patient's body, for example a device having a conductive body 400 overlain with an insulating layer such as a coating 402 defining more than one current-delivering window 408, and applying energy through the device to treat a region of tissue. The method may include additional steps including, but not limited to, positioning the device using fluoroscopic imaging (or other medical imaging techniques such as CT, MRI and ultrasound) or other forms of guidance, repositioning the device where necessary, stimulating neural tissue by applying, for example, low-frequency energy, cooling at least a portion of the device, adjusting the electrical conductivity of the device, measuring an electrical or physiological parameter (e.g. tissue temperature, impedance, pressure, etc.) and adjusting an aspect of the treatment procedure based on the measured parameter.

The step of inserting the device into the body may include the additional steps of: measuring or detecting a property of a tissue, such as impedance, applying an anesthetic or antiseptic solution to a tissue, or creating an incision or puncture in a tissue.

The step of applying energy through the device to treat the tissue may include, but is not limited to: applying energy in a monopolar configuration so that energy travels from the device to a reference electrode located at some distance away from the device; applying energy in a bipolar configuration so that energy travels from the device to another probe or electrode or from another probe or electrode to the device; applying energy in a bipolar configuration from one region of the device to a separate, electrically-isolated region of the device; applying energy in a multi-polar configuration; applying energy in any of the above manners whereby the size, impedance, or position of the active or return electrodes is dynamically changed; applying energy continuously; applying energy periodically (i.e. in a pulsed manner), according to a set duty cycle; applying energy periodically according to feedback generated by measurement of tissue or treatment parameters; automatically varying the voltage or current of energy delivered based on pre-set parameters, or measured parameters; and manually varying the voltage or current of energy delivered.

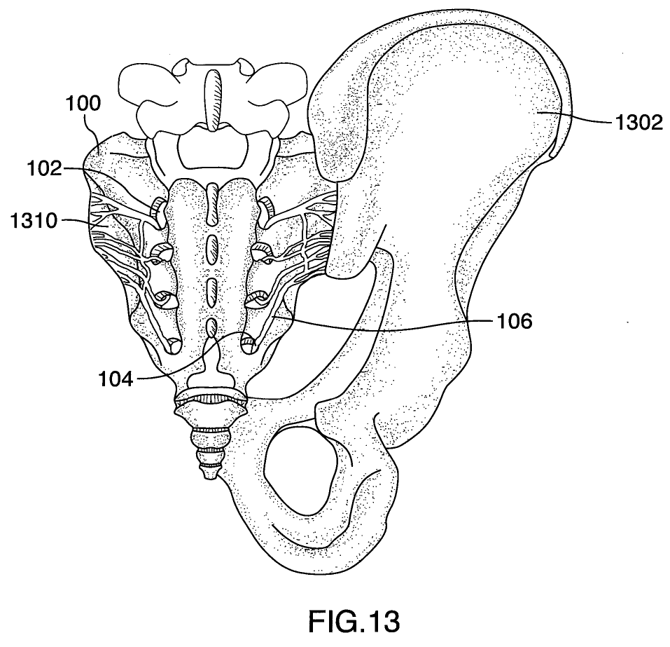

One specific application of a device of the present invention is for the treatment, prevention, or diagnosis of pain emanating from the sacroiliac (SI) region. Pain associated with the Si region has been referred to in the literature as sacroiliac syndrome, sacroiliac joint dysfunction or sacroiliac joint complex (SIJC) pain, amongst other terms, and, for clarity, will be referred to throughout this description as sacroiliac joint syndrome (SIJS). Other symptoms of SIJS that may be treated and/or reduced by embodiments of the method aspect of the present invention include stiffness and tingling. Referring to FIG. 13, the SI joint 1310 is the joint between the sacrum 100, a large bone at the base of the spine composed of five fused vertebrae, and the ilium 1302 of the pelvis. SI joint 1310 is a relatively immobile joint, serving to absorb shock during locomotion. The structure of the SI joint varies significantly between individuals but generally comprises an articular cartilaginous surface, a ligamentous aspect and, in most cases, one or more synovial recesses. Historically, it was believed that SI pain was referred, and that the joint itself was not innervated, however, it has recently become accepted that nerves do enter the joint. Though the specific pathways of innervation have not yet been elucidated, the nerves responsible for SI pain are thought to comprise, at least in part, nerves emanating from the sacral dorsal plexus, the network of nerves on the posterior surface of the sacrum, extending from the sacral nerves 104, also referred to as the poseterior primary rami, that exit the sacral foramina (posterior sacral foramen) 102. Diagnostic criteria for SIJS include the following: (1) pain in the region of the SI joint with possible radiation to the groin, medial buttocks, and posterior thigh, (2) reproduction of pain by physical examination techniques that stress the joint, (3) elimination of pain with intra-articular injection of local anesthetic and (4) an ostensibly morphologically normal joint without demonstrable pathognomonic radiographic abnormalities. While mechanical support devices exist for the alleviation of pain, there is currently no standardized method or apparatus for the treatment of SIJS.

In order to treat or prevent SI pain, a procedure may be followed according to a method of the present invention for lesioning posterior primary rami 104 that innervate the SI joint, while preventing the lesioning of areas surrounding the posterior primary rami, for example the posterior sacral foramen. The posterior primary rami may lie at or adjacent to the location where they exit the sacral foramina 102, for example an area lateral to the foramen, between 2 o'clock and 6 o'clock on the right-hand side and between 6 o'clock and 10 o'clock on the left hand side of the sacrum (when the foramen is viewed as a clock face). It should be noted that the locations at which the posterior primary rami exit the foramen may vary between individuals and the area described above, i.e. between 2 o'clock and 6 o'clock, is not intended to be limiting.

In one embodiment, a procedure may proceed as follows: The patient should be placed in the prone position on a fluoroscopy table. An introducer 1100 may then be utilized to position the device within the patient's body. For example, under image guidance, an introducer 1100 should be inserted caudally, inferior and lateral to the posterior sacral foramen of S3. The tip of introducer 1100 should point cranially, in line with the inferior portion of the lateral sacral crest. Probe 300 should then be inserted into introducer 1100 and guided to navigate cranially and trace the curvature of the posterior sacral foraminal aperture (PSFA) (lateral to posterior sacral foramen 102 and medial to the lateral sacral crest), so that current-delivering windows 408 are facing away from posterior sacral foramen 102. This positioning may allow for the lesioning of the posterior primary rami, while preventing the formation of a lesion within the foramen 102. This specific positioning of probe 300 may be facilitated by providing a steerable probe 300, a curved probe 300 and/or a curved introducer 1100, as has been described above.

Once probe 300 is in position, RF energy may be delivered to create at least one lesion along sacrum 100, adjacent the lateral sacral crest, while posterior sacral foramen 102 is at least partly shielded from RF energy by insulating material located along probe 300. Thus, in such embodiments, the target site comprises at least a portion of a sacral neural crescent. Energy may be delivered using an RF signal having a voltage up to about 500V, current up to about 5 amperes, a frequency of about 200 kHz to about 10 MHz and an application interval of about 5 seconds to about 30 minutes; for tissue in the sacroiliac region, the signal may, in some embodiments, have a voltage ranging between about 10V and about 200V, a frequency of about 400 to about 550 kHz, an application interval of about 1 to about 10 minutes, and a power of about 1 to about 20 Watts. In certain embodiments, energy may also be delivered through introducer 1100 in order to create an effective lesion. It should be clear to those skilled in the art that an opposite approach (for example, inserting a device cranially as opposed to caudally) of the positioning method described herein may be used as well and the invention is not intended to be limited to one specific approach or at one specific level of the sacrum.

Probe 300 may be able to create a lesion, for example a strip lesion, adjacent the sacrum, in order to treat as many neural structures of the dorsal sacral plexus as possible within a single treatment procedure. For example, at least two lateral branches 106 of a sacral nerve 104 may be treated during such a procedure. However, the ability to create a strip lesion is not necessary in some embodiments. Rather, various probes, capable of producing lesions of various shapes and sizes, may also be used in conjunction with this aspect of the present invention and the invention is not limited in this regard.

In some embodiments of the present invention, a supporting or stabilizing apparatus or device may be used to help prevent inadvertent movement of the probe(s) and/or introducer(s). For example, the supporting or stabilizing apparatus (i.e. a means for supporting or stabilizing) may, for example, take the form of a frame for fixing the probe(s) and/or introducer(s) in a desired position. During the step of inserting the probe(s), the position of the probe(s) or introducer(s) may be visualized and/or monitored, for example by using fluoroscopy or other imaging modalities. If fluoroscopy is used, visualization may be improved by incorporating one or more radio-opaque markers onto one or more of the probe(s) or introducer(s). In some embodiments, radio-opaque markers may be incorporated onto a distal region of the probe(s) in order to determine the distance that the probes are extending out of the introducer(s). In addition, visual depth markers may be used to help determine the position of the probe(s) or introducer(s) within the body. Furthermore, positioning may be confirmed by measuring the impedance of tissue at the location of the probe(s) or introducer(s), as is known in the art. In some embodiments, positioning may not be verified using these means and a user may rely in whole or in part on his knowledge of a patient's anatomy in order to accurately place the device(s).

In some embodiments, the probe(s) used in this method aspect of the present invention may be operable to treat a plurality of neural structures without the need for one or more of removal of the probe(s), reinsertion of the probe(s) or repositioning of the probe(s). For example, at least two branches 106 of a sacral nerve 104 may be treated. These two branches may comprise, for example, two or more branches of the same sacral nerve. Treatment of two or more branches of the same sacral nerve may be facilitated by delivering energy to at least a portion of a sacral neural crescent, as described above and as shown in FIG. 1. In such an embodiment, the method may comprise the step of delivering energy to a sacral neural crescent to create a substantially homogeneous lesion to treat at least two branches of a sacral nerve passing through the sacral neural crescent. Alternatively, these two branches may comprise at least one lateral branch from one sacral nerve and at least one lateral branch from a different sacral nerve.

One way of achieving this effect is to create a single strip lesion at a desired location, wherein said single strip lesion may be of sufficient size so as to affect multiple neural structures. As has been mentioned, the probe(s) may be operable to create such a lesion during the course of a single energy delivery step or while the probe remains in a substantially static position, i.e. without the need for one or more of removal of the probe(s), reinsertion of the probe(s) or repositioning of the probe(s). Thus, as used herein, a ‘single energy delivery step’ may refer to a single temporally continuous period of energy delivery. For example, creating a strip lesion in a single energy delivery step may allow for movement of one or more portions of the probe to create the strip lesion, as long as the delivery of energy is maintained during any such probe movement.

As a feature of this aspect of the present invention, some embodiments may further comprise a step of moving the probe(s) to another location within the tissue if the user so desires. The probe(s) may be moved before, during, or after the step of delivering energy, and may be moved one or more times. The step of moving the probes may comprise one or more of the following actions: applying a force to bend the probe within the tissue (wherein the probe may thus be described as a ‘steerable’ probe), moving the probe intact within the tissue, removing the probe intact from the tissue, reinserting the probe into the tissue and moving one or more parts of the probe (for example, extending or retracting a segmented probe telescopically) to move the position of one or more functional elements within the tissue. For example, one such embodiment of this method aspect may comprise a step of repositioning a probe at another sacral neural crescent, wherein energy may be delivered to treat tissue within the other sacral neural crescent.

As a further feature of this aspect of the present invention, the method may further comprise one or more steps of modifying a treatment procedure in response to one or more measured parameters. These measured parameters may include, but are not limited to, temperature, position of the probe(s) or impedance, or any combination thereof. For example, if a temperature measurement is determined to be outside of a desired range, a treatment procedure may be modified by, for example, altering the amount of energy delivered by the generator, modifying or modulating the one or more means for cooling in some way, or terminating the procedure. As another example, the amount of energy delivered by the generator may be modified based on the position of the one or more probes (for example, depending on the distance between a probe and the target treatment site or on the distance between the probes themselves when more than one probe is used). In such embodiments, a feedback system may be incorporated directly into the energy source so that any modification of a treatment procedure in response to a measured parameter may occur automatically. In other embodiments, there may not be an automatic feedback system in place, in which case a user may manually modify a treatment procedure in response to a measured parameter. In addition to modifying a treatment procedure based on measured parameters, this invention also provides for a step of determining the initial parameters to be used in a treatment procedure (for example, the initial maximum power level or tissue temperature, temperature ramp rate, etc.) using information that is known about the particular tissue to be treated. For example, if pre-treatment testing reveals specific information about the sacrum of a particular patient (this information may include, but is not limited to: the topology of the sacrum, location of specific nerves, etc.), that information may be used to decide on what parameters to use initially for the treatment procedure.

In an alternate embodiment, a lesion may be created in two parts. Introducer 1100, which, in this embodiment, has an aperture located at or near its distal end, is inserted towards a posterior sacral foramen 102, with the aperture pointing cranially. Probe 300 is then inserted into introducer 1100 and advanced cranially through the aperture such that current-delivering windows 408 are facing away from posterior sacral foramen 102. Once probe 300 is in position, RF energy is delivered to create a first portion of a lesion along sacrum 100. Probe 300 is then retracted into or removed through introducer 1100, at which point introducer 1100 is rotated around its longitudinal axis so that the aperture points caudally. Probe 300 (which may be the same probe, or may be a different probe) is then inserted into introducer 1100 and advanced caudally through the aperture such that current-delivering windows 408 are again facing away from posterior sacral foramen 102. Once probe 300 is in position, RF energy is delivered to create a second portion of the lesion. Either of probe 300 and introducer 1100 may be rotated any number of times after the initial insertion adjacent a single posterior sacral foramen 102, and the above steps may be repeated adjacent multiple posterior sacral foramina 102.

In another embodiment, probe 300 may be used to create a lesion at a target site along the anterior or posterior portions of the SI joint margin, for example to allow for a lesion along substantially the entire length of the joint capsule, while preventing the formation of a lesion in the area surrounding the joint margin. For example, probe 300 may be inserted cranially towards the posterior portion of the joint margin. Probe 300 may be positioned such that the current-delivering windows lie substantially along the joint margin, with current-delivering windows 408 facing anteriorly. Once probe 300 is in position, RF energy is delivered to create of a lesion along the joint margin, while the area around the joint margin is shielded from the RF energy by the insulating material of probe 300. Alternatively, probe 300 may be used to create a lesion at a target site within SI joint 1310 wherein energy may be delivered to a desired portion of the SI joint while shielding another portion of the SI joint from energy delivery. For example, it may be desirable to prevent energy from being delivered to regions of the SI joint 1310 containing collagen in order to limit or prevent the shrinkage or tightening of the collagen within the joint.

In yet another embodiment of a method of the present invention, an incision is made in the tissue overlaying sacrum 100, allowing insertion of an introducer adjacent the posterior sacral foramen 102. Incising the tissue allows for the insertion of a rigid introducer 1100 having a substantial curve into the tissue without risking unwanted tissue damage or incorrect positioning, which can result from the navigation of a device along a lengthy curved path within the tissue. Probe 300 is then inserted into introducer 1100 and advanced therethrough adjacent posterior sacral foramen 102 so that current-delivering windows 408 are facing away from posterior sacral foramina 102 and slightly anteriorly, towards sacrum 100. Once probe 300 is in position, RF energy is delivered to create at least one strip lesion along sacrum 100, while preventing the formation of a lesion at the posterior sacral foramina 102.

Alternatively, the methods of treatment described herein may be practiced without an introducer, wherein probe 300 is inserted directly to the treatment site. In some embodiments, introducer 1100 and probe 300 may be electrically coupled and both may have current-delivering windows, so that the step of delivering energy to the tissue can result in the creation of a lesion adjacent at least a portion of introducer 1100, as well as adjacent probe 300.

According to yet another aspect of the invention, the step of advancing or navigating probe 300 through the tissue adjacent posterior sacral foramen 102 involves changing the shape of probe 300 by applying energy to a guide wire or hinge or other mechanism in order to bend or otherwise alter the shape of probe 300. The shape of probe 300 may also be changed passively in devices wherein probe 300 is manufactured from a material having a shape memory (e.g. Nitinol), or wherein probe 300 is statically affixed to introducer 1100 with a guide wire, tension being created in said wire when probe 300 is advanced out of introducer 1100.

In a further embodiment, the present invention may be used to diagnose SI pain, whereby probe 300 is inserted and positioned in accordance with any of the embodiments described hereinabove, and energy is delivered at a low frequency sufficient to reproduce a patient's pain. If the patient's pain is reproduced upon delivery of energy, the pain may be diagnosed as emanating from the SI region, and the patient may be diagnosed with sacroiliac joint syndrome. If the patient's pain is not reproduced, an alternate diagnosis may be pursued.

In other embodiments, methods in accordance with the present invention may be performed at other regions within a patient's body. For example, in some cases, it may be desired to ablate a sensory nerve that is in close proximity to a motor nerve. If probe 300 is inserted such that the current-delivering windows 408 are pointing towards the sensory nerve and away from the motor nerve, the sensory nerve may be ablated without damaging the motor nerve. In another example, it may be desired to ablate a nerve that lies along a bony surface, for example along the articular process of a vertebra. In this case, probe 300 may be inserted such that current-delivering windows 408 are facing the surface of the bone. Therefore the nerve that lies along the bony surface may be ablated, while avoiding damage to the surrounding tissue.

The embodiments of the invention described above are intended to be exemplary only. The scope of the invention is therefore intended to be limited solely by the scope of the appended claims.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

We claim:

1. A method of delivering energy to a sacroiliac region of a patient's body, through a device having at least one electrically insulated region along a portion of a circumference of the device and at least one current delivering region along a remaining portion of the circumference of the device comprising, in any order, the steps of:
positioning the electrically insulated region and the current delivering region within the sacroiliac region of a patient's body such that the current delivering region is electrically exposed to a target site and at least a portion of the electrically insulated region shields another site; and
delivering energy to the device;
whereby energy is delivered substantially towards the target site and substantially prevented from being delivered to the shielded site.

2. The method of claim 1, wherein the shielded site comprises a posterior sacral foramen.

3. The method of claim 2, wherein the target site comprises at least a portion of a sacral neural crescent.

4. The method of claim 1, wherein the target site comprises at least a portion of a sacroiliac joint margin.

5. The method of claim 1, wherein the target site is located within a sacroiliac joint.

6. The method of claim 1, wherein energy is delivered in order to reduce one or more symptoms of sacroiliac joint syndrome.

7. The method of claim 6, wherein said one or more symptoms of sacroiliac joint syndrome comprises one or more of: pain, stiffness and tingling.

8. The method of claim 1, wherein energy is delivered in order to alter the structure or function, or both, of neural tissue.

9. The method of claim 8, wherein the step of delivering energy comprises delivering energy sufficient to ablate one or more neural structures.

10. The method of claim 8, wherein the target site includes at least one sacral nerve and wherein energy is delivered in order to treat the at least one sacral nerve.

11. The method of claim 8, wherein the target site includes at least two branches of a sacral nerve and wherein energy is delivered to treat the at least two branches of the sacral nerve.

12. The method of claim 1, wherein the step of delivering energy comprises delivering energy selected from the group consisting of:
electromagnetic energy ranging from radio-frequency energy to optical energy, thermal energy and microwave energy.

13. The method of claim 12, wherein the step of delivering energy comprises delivering radio-frequency energy.

14. The method claim 1, further comprising the steps of repositioning the device at a second location within the sacroiliac region of a patient's body and delivering energy to the device.

15. The method of claim 1, wherein energy is delivered in order to create a strip lesion.

16. The method of claim 1, wherein energy is delivered in order to create a substantially homogeneous lesion.

17. The method of claim 1, wherein the device comprises current delivering windows separated by a material that is at least a partial electrical insulator.

18. The method of claim 17, wherein at least some of the current delivering windows are electrically isolated from each other.

19. The method of claim 1, wherein the device comprises at least one marker for determining the position of the current delivering region.

20. The method of claim 1, further comprising a step of measuring at least one parameter selected from the group consisting of temperature, pressure and impedance.

21. The method of claim 1, further comprising a step of cooling at least a portion of the device.

22. The method of claim 1, wherein the step of positioning the device utilizes an introducer.

23. The method of claim 1, wherein the device is non-linear.

24. The method of claim 23, wherein the device comprises at least one curve.

25. The method of claim 24, wherein the curve has a central angle of between about 110 degrees and about 130 degrees, and a radius of curvature between about 5 millimeters and about 15 millimeters.
